(19) United States
(12) Patent Application Publication
(10) Pub. No.: US 2014/0343685 A1
(43) Pub. Date: Nov. 20, 2014

(54) FEMORAL COMPONENT FOR A HIP PROSTHESIS

(71) Applicants: Chitranjan S. Ranawat, Alpine, NJ (US); Amar S. Ranawat, New York, NY (US); Leslie L. Jacobs, Far Hills, NJ (US); Morteza Meftah, New York, NY (US)

(72) Inventors: Chitranjan S. Ranawat, Alpine, NJ (US); Amar S. Ranawat, New York, NY (US); Leslie L. Jacobs, Far Hills, NJ (US); Morteza Meftah, New York, NY (US)

(21) Appl. No.: 14/282,402

(22) Filed: May 20, 2014

Related U.S. Application Data
(60) Provisional application No. 61/825,158, filed on May 20, 2013.

(51) Int. Cl. A61F 2/36

(52) U.S. Cl. CPC A61F 2/3662; A61F 2002/3678
USPC 623/23.35

(57) ABSTRACT

A femoral component for a hip prosthesis has a proximal end for disposition at a superior position in a femur and a distal end opposite the proximal end. The femoral component has opposite medial and lateral sides and opposite anterior and posterior faces extending between the medial and lateral sides. At least a proximal part of the femoral component has a first superior-to-inferior taper so that a medial to lateral dimension decreases gradually from the proximal end toward the distal end, a second superior-to-inferior taper so that an anterior to posterior dimension of the femoral component decreases gradually from the proximal end toward the distal end and a lateral-to-medial taper so that an anterior to posterior dimension of the femoral component decreases gradually from the lateral side toward the medial side.

This application claims priority on U.S. Provisional Application No. 61/825,158 filed on May 20, 2013.

### BACKGROUND OF THE INVENTION

1. Field of the Invention

The invention relates to the femoral component of a hip prosthesis.

2. Description of the Related Art

A hip replacement prosthesis includes a femoral component and an acetabular component. The femoral component has an elongated stem for placement in the intramedullary canal of the femur. A neck extends from the proximal end of the stem and a spherical head is attached to the proximal end of the trunnion at the neck.

The femur is prepared by removing the head and neck of the natural femur and preparing the intramedullary canal for receiving the stem of the femoral component. The size and other physiological characteristics of the femur vary widely from one patient to another. Accordingly, the size and shape of the femoral component must be selected to reflect the size and physiological characteristics of the patient. Ideally, the proximal part of the stem of the femoral component of the prosthesis should be supported by the intramedullary canal of the femur. Distal parts of the stem of the femoral component of the prosthesis help to guide the stem into a neutral axis of the intramedullary canal, but preferably should not provide load transfer from the head and neck of the femoral component to the femur of the patient. An improper fit and improper engagement of the distal parts of the stem of the femoral component with the cortical bone of the intramedullary canal can cause proximal femoral loosening. The movement of the proximal stem of the femoral component can be compared to the movement of a windshield wiper and affect the mechanics of the prosthetic joint. Furthermore, the engagement of the distal portion of the stem with the cortical bone of the natural femur will shield the more proximal metaphyseal/diaphyseal area of the femur from a load bearing function, and this stress shielding can lead to deterioration of the metaphyseal/diaphyseal area of the femur and lead to loosening of the femoral component.

The invention was made in view of the above-described potential problems with the prior art femoral component of a hip prosthesis. Accordingly, it is an object of the subject invention to optimized contact and accurate mounting of the femoral component of a hip prosthesis in the proximal part of the intramedullary canal of the femur. It is another object of the subject invention to prevent pivoting movement of the femoral component about an axis near the distal end of the stem. It is a further object of the subject invention to prevent a stress shielding of the metaphyseal/diaphyseal area of the femur, thereby preserving bone in this region of the femur

### SUMMARY OF THE INVENTION

The invention relates to the femoral component of a hip prosthesis. The femoral component includes a stem to be mounted in the intramedullary canal and a neck projecting proximally from the proximal end of the stem. In this regard, the distal end of the stem is considered to be the end of the stem at the more inferior position, while the proximal end of the stem is substantially at the superior end of the surgically prepared femur and substantially at the superior entry to the intramedullary canal. The neck of the femoral component will extend at an angle to the axis defined by distal portions of the stem, and typically at an angle of approximately 130°. The length of the stem will vary in accordance with the size and other physiological characteristics of the patient.

For purposes of explaining the invention, the proximal part of the stem of the femoral component will be considered to have a superior region extending proximally from a position aligned with a center the lesser trochanter in a direction perpendicular to the axis of the stem and continuing in a superior direction to the superior end of the surgically prepared femur (substantially at the greater trochanter). The proximal part of the stem of the femoral component will also be considered to have an inferior region extending in an inferior direction from the superior region of the proximal part of the stem by a distance substantially equal to the axial length of the superior region of the proximal part of the stem. The distal or inferior end of the proximal part of the stem typically will be about 20 to 35 mm below the lesser trochanter, depending on the size of the femur. The superior and inferior regions of the proximal part of the stem are configured to define a plurality of wedge-shaped tapers, as described herein, for securely engaging the metaphyseal/diaphyseal endosteal bone tissue near the proximal end of the femur to substantially prevent axial, torsional and bending forces within the proximal intramedullary canal, to control the location of the axial loads of the femoral component on the natural femur and to reduce bending moments in the stem.

The plurality of wedge-shaped tapers on the proximal part of the stem include a superior-to-inferior taper so that medial-to-lateral dimensions of the proximal part of the stem become gradually smaller from the superior end of the superior region of the proximal part of the stem to the inferior end of the inferior region of the proximal part of the stem. Additionally, the wedge-shaped superior-to-inferior taper is configured so that anterior-to-posterior dimensions of the stem become gradually smaller from the superior end of the superior region of the proximal part of the stem to the inferior end of the inferior region of the proximal part of the stem. The superior-to-inferior taper preferably is approximately 4°.

The plurality of wedge-shaped tapers on the proximal part of the stem further include a medial-to-lateral taper so that an anterior-to-posterior dimension of the proximal part of the stem gradually increases from a minimum at the medial part of the stem to a maximum at the lateral part of the stem. The medial-to-lateral taper preferably is in a range of 6°-7° and varies with the axial position along the stem, as explained further herein.

The plurality of wedge-shaped tapers on the proximal part of the stem may further include an anterior-to-posterior taper so that a medial-to-lateral dimension of the proximal part of the stem gradually increases from a minimum at the posterior part of the stem to a maximum at the anterior part of the stem. The anterior-to-posterior taper preferably is in a range of 3°-5°.

### BRIEF DESCRIPTION OF THE DRAWINGS

FIG. 1 is a front elevation view of a femoral component of a hip prosthesis in accordance with the subject invention.

FIG. 2 is a front elevation view similar to FIG. 1, but without the roughened surface texture on proximal portions of the femoral component.

FIG. 3 is a cross-sectional view taken along line 3-3 in FIG. 1.

FIG. 4 is a cross-sectional view taken along line 4-4 in FIG. 1.

FIG. 5 is a cross-section of view taken along line 5-5 in FIG. 1.

FIG. 6 is a cross-sectional view taken along line 6-6 in FIG. 1.

FIG. 7 is a side elevation view looking in a medial-to-lateral direction and taken from the right side of FIG. 1.

FIG. 8 is a side elevation view looking in a lateral-to-medial direction and taken from the left side in FIG. 1.

FIG. 9 is a top plan view of the femoral component.

FIG. 10 is a bottom plan view of the femoral component.

FIG. 11 is a cross-sectional view showing the femoral component implanted in a femur.

### DETAILED DESCRIPTION OF THE PREFERRED EMBODIMENT

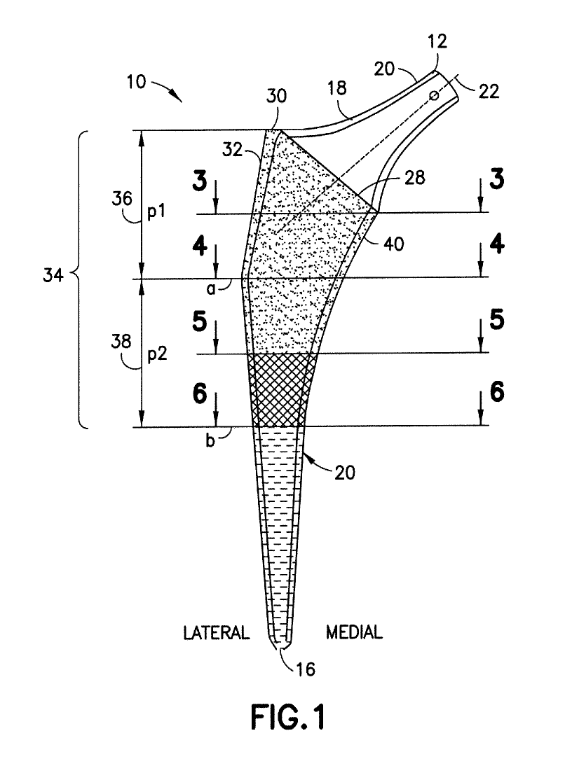

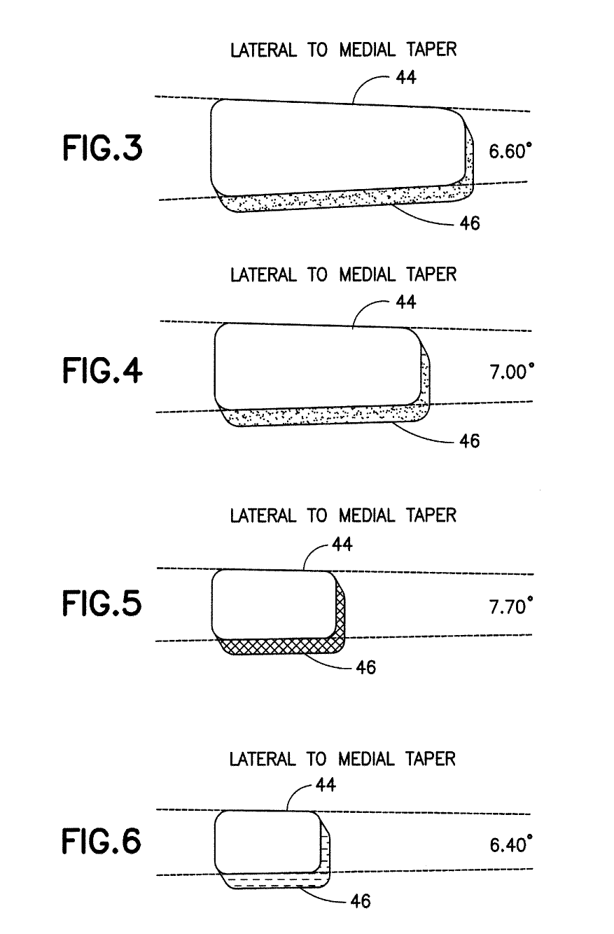

A femoral component of a hip prosthesis in accordance with the invention is identified generally by the numeral 10 in FIGS. 1-8. The femoral component 10 has a proximal end 12 and a distal end 16. A neck 18 extends distally from the proximal end 16 and includes a tapered cylinder or frustum 20 adjacent the proximal end 16. A neck axis 22 extends substantially along the neck 18 substantially concentrically with an axis defined by the frustum 20. A spherical head (not shown) will be mounted on the frustum 20 and will be engageable with the acetabular component (not shown) of the hip prosthesis.

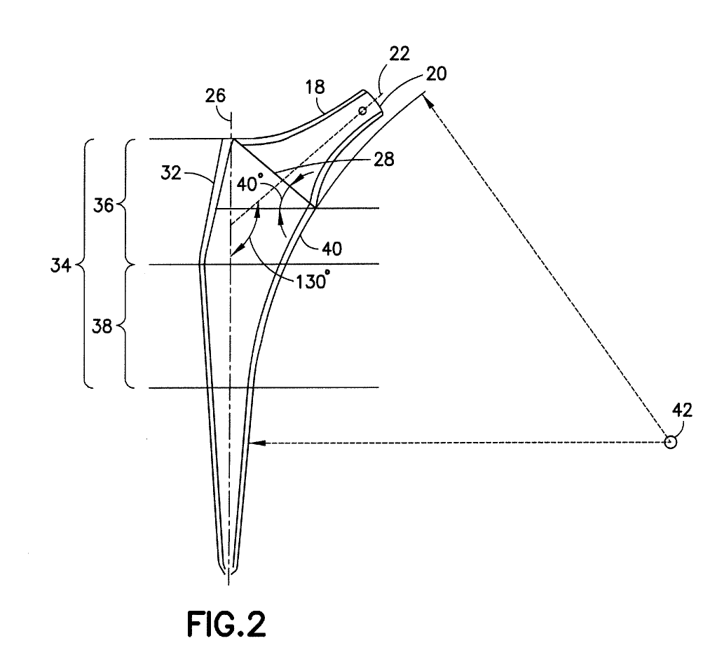

A stem 24 extends from the neck 18 to the distal end 16 of the femoral component 10. A stem axis 26 extends substantially centrally along distal parts of the stem 22 and an extension of the stem axis 26 intersects the neck axis 22 at an angle “A” of approximately 130°, as shown in FIG. 2. The stem 24 joins the neck 18 at a plane 28 that intersects a plane perpendicular to the stem axis 26 at an angle “B” of approximately 40°.

FIG. 2 shows a line “a” extending substantially perpendicular to the stem axis 26 at a distance p1 from the proximal end 30 of the stem 24. In this regard, the proximal end 30 of the stem 24 is disposed along a lateral side 32 of the stem 24 and at a position that will be substantially adjacent the greater trochanter of the natural femur. The line “a” will pass substantially through a center of the lesser trochanter of the natural femur. FIG. 2 also shows a line “b” extending substantially perpendicular to the stem axis 26 at a distance p2 distally from the line “a.” The distance p2 is substantially equal to the distance p1. The area of the stem 24 extending from the proximal end 30 of the stem 24 to the line “b” is referred to herein as the proximal part 34 of the stem 24. The area of the proximal part 34 of the stem 24 between the proximal end 30 of the stem 24 and the line “a” will be referred to herein as the superior region 36 of the proximal part 34 of the stem 24. The area of the proximal part 34 of the stem 24 between the line “a” and the line “b” will be referred to herein as the inferior region 38 of the proximal part of the stem 24. The outer surface of the superior region 36 of the proximal part 34 of the stem 24 has a porous coating of hydroxyapatite or other osteoinductive/osteoconductive agent to achieve a secure non-adhesive affixation of the stem 24 in the intramedullary canal of a femur. The outer surface of the inferior region 38 of the proximal part 34 of the stem 24 preferably has a roughness of 180 Ra. The outer surface of the distal part of the stem below the line “b” preferably has a satin finish with a roughness of approximately 30 Ra. The more smooth finish at more distal positions on the stem 24 avoids affixation at these locations and facilitates any surgical revision that may be necessary. On the other hand, the rougher surface more proximal positions achieves secure proximal affixation with a desired load transfer to proximal portions of the femur.

The stem 24 includes a proximal-to-distal taper as shown most clearly in FIGS. 1, 2, 7 and 8. More particularly, the stem 24 includes a medial side 40 opposite the lateral side 32 and the taper is oriented so that a medial-to-lateral distance between the medial side 40 and the lateral side 32 gradually decreases from the proximal end 30 to the distal end 16 of the femoral component 10. The proximal-to-distal taper is achieved partly by a concave curve 42 extending from a location where the medial side 40 of the stem 24 intersects the plane 28 between the stem 24 and the neck 18 to a location distally of the line “b”. The part of the medial side 40 extending from the concave curve 42 to the distal end 16 of the femoral component 10 may extend substantially linearly and tangent to the concave curve 42. The medial to lateral taper at the point of tangency between the concave curve 42 and more distal parts of the stem 24 preferably is about 8° and preferably is substantially symmetric with the axis 26 of the stem 24.

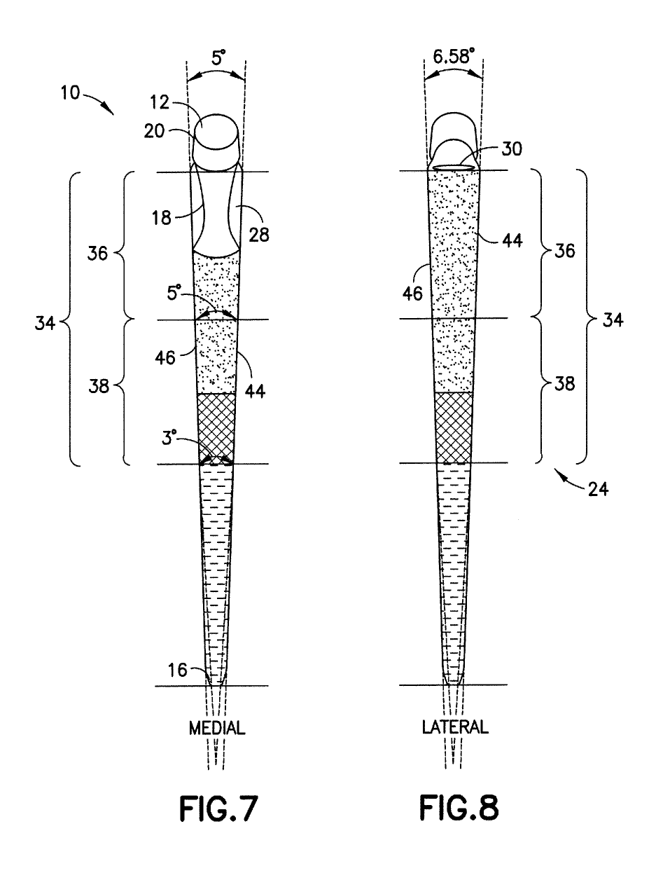

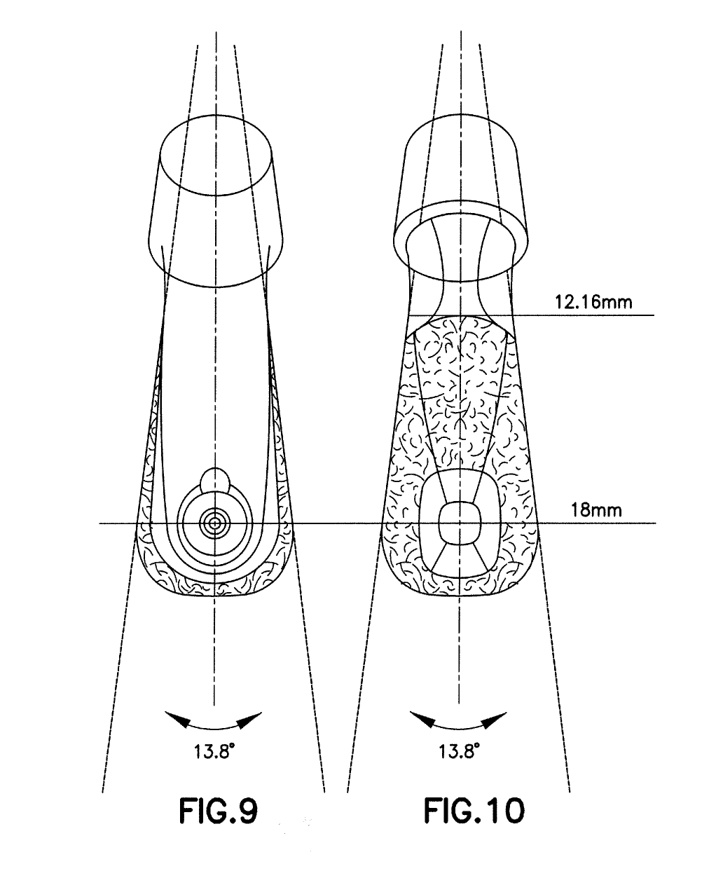

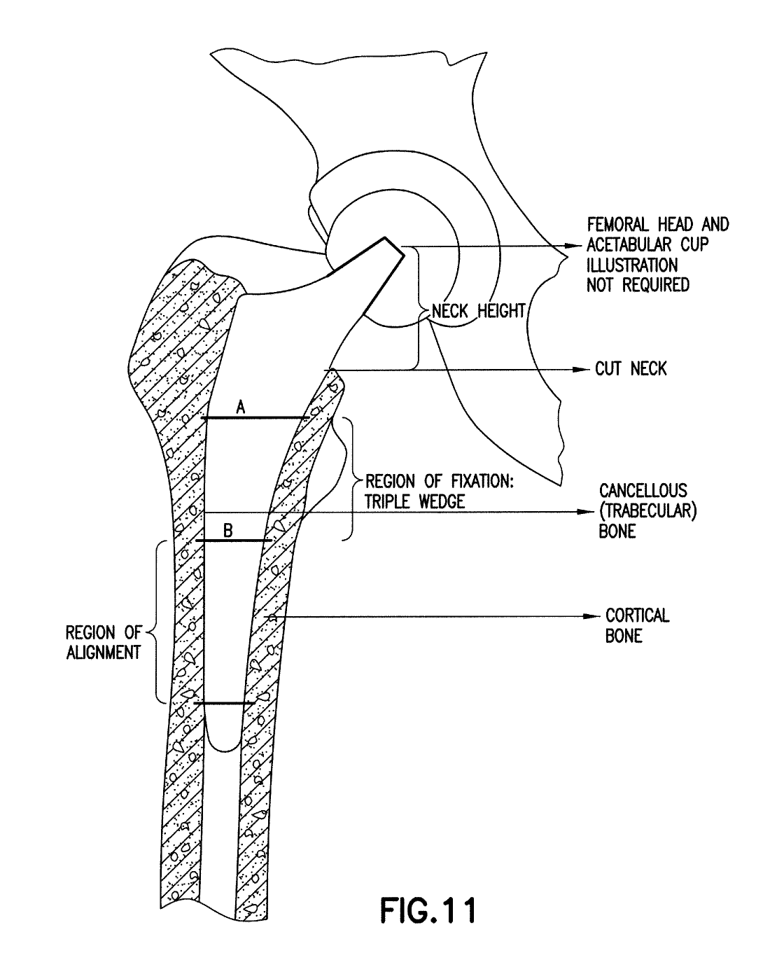

The proximal-to-distal taper also is configured so that an anterior-posterior dimension gradually decreases from the proximal end 30 the stem 24 to the distal end 16 of the femoral component 10. More particularly, as shown in FIGS. 7 and 8, the stem 24 includes an anterior surface 44 and a posterior surface 46. The proximal-to-distal taper is configured so that a distance between the anterior surface 44 and the posterior surface 46 gradually decreases along the stem 24 toward the distal end 16. The proximal-to-distal taper between the anterior surface 44 and the posterior surface 46 preferably is angularly larger at locations closer to the proximal end of the stem 24. For example, the superior region 36 of the proximal part 34 of the stem 24 preferably has the anterior surface 44 and the posterior surface 46 aligned at an angle of about 5°. The inferior region 38 of the proximal part 34 of the stem 24 preferably has the anterior surface 44 and the posterior surface 46 aligned at an angle of about 3°.

The proximal part 34 of the stem 24 also has a medial-to-lateral taper as shown most clearly in FIGS. 3-6. More particularly, the medial-to-lateral taper is oriented so that dimensions between the anterior surface 44 and the posterior surface 46 of the stem 24 become gradually greater at distances farther from the medial side 40 and closer to lateral side 32. The medial-to-lateral taper of the proximal part 34 of the stem 24 becomes gradually less at more distal positions on the proximal part 34 of the stem 24. In this regard, and as shown in FIG. 3, the medial-to-lateral taper of the superior region 36 of the proximal part 34 of the stem 24 preferably is approximately 5° at locations approximately midway between the proximal end 30 of the stem 24 and the line “a” between the superior region 36 and the inferior region 38 of the proximal part 34 of the stem 24. As noted above, the line “a” is approximately aligned with the center of the lesser trochanter. The medial-to-lateral taper preferably is approximately 3° at the line “a” and is reduced to approximately 2° along the inferior region 38 of the proximal part 34 of the stem 24.

The stem 24 also includes an anterior-to-posterior taper as shown in FIGS. 3-6. More particularly, a distance between the lateral side 32 and the medial side 40 of the stem 24 decreases gradually from the anterior surface 44 to the posterior surface 46. The anterior-to-posterior taper preferably is approximately 5° along the superior region 36 of the proximal part 34 of the stem 24 and gradually changes to approximately 3° along the inferior region 38 of the proximal part 34 of the stem 24.

As described above, the femoral component 10 has a stem 24 with a tapered wedge-shape in three directions, namely a proximal-to-distal taper, a medial-to-lateral taper and an anterior-to-posterior taper. Each of the three tapers preferably is slightly greater at more superior positions along the proximal part 34 of the stem 24. As a result, secure proximal affixation of the femoral component 10 in the intramedullary canal of the femur is achieved reliably, thereby preventing tilting of the stem 24 about positions close to the distal end 16, avoiding bending of the stem 24 and preventing stress shielding at the superior end of the femur.

What is claimed is:

1. A femoral component for a hip prosthesis having a proximal end for disposition at a superior position in a femur and a distal end opposite the proximal end, the femoral component having opposite medial and lateral sides, an anterior face and a posterior face substantially opposite one another and extending between the medial and lateral sides, at least a proximal part of the femoral component having a first superior-to-inferior taper so that a medial to lateral dimension of the femoral component decreases gradually from the proximal end toward the distal end, a second superior-to-inferior taper so that an anterior to posterior dimension of the femoral component decreases gradually from the proximal end toward the distal end and a lateral-to-medial taper so that an anterior to posterior dimension of the femoral component decreases gradually from the lateral side toward the medial side.

2. The femoral component of claim 1, wherein the lateral-to-medial taper and the first and second superior-to-inferior tapers extend from the proximal end of the femoral component to a position substantially aligned with the lesser trochanter of the femur.

3. The femoral component of claim 2, wherein the lateral-to-medial taper and the first and second superior-to-inferior tapers extend from the proximal end of the femoral component for a distance substantially equal to twice a distance from the proximal end of the femoral component to the position substantially aligned with the lesser trochanter of the femur.

4. The femoral component of claim 1, wherein the second superior-to-inferior taper defines an angle of taper of between about 3° to about 5°.

5. The femoral component of claim 5, wherein the angle of taper defined by the second superior-to-inferior taper becomes gradually smaller at positions farther from the proximal end of the femoral component.

6. The femoral component of claim 1, wherein the lateral-to-medial taper defines a lateral-to-medial taper angle of approximately 5° to approximately 3°.

7. The femoral component of claim 1 wherein the medial surface of the femoral component defines a continuous concave curve extending at least along a portion of the femoral component having the first superior-to-inferior taper.

8. The femoral component of claim 1, further comprising a surface treatment for promoting non-adhesive affixation extending along areas of the femoral component having the lateral-to-medial taper and the first and second superior-to-inferior tapers.
